(19) United States
(12) Patent Application Publication
(10) Pub. No.: US 2007/0203416 A1
(43) Pub. Date: Aug. 30, 2007

(54) Blood pressure cuffs

(76) Inventor: Andrew Lowe, Meadowbank (NZ)

Correspondence Address: MILDE & HOFFBERG, LLP, 10 BANK STREET, SUITE 460, WHITE PLAINS, NY, 10606, US

(21) Appl. No.: 11/495,279

(22) Filed: Jul. 28, 2006

Related U.S. Application Data
(60) Provisional application No. 60/777,418, filed on Feb. 28, 2006.

(51) Int. Cl. A61B 5/02

(52) U.S. Cl. 600/485

(57) ABSTRACT

A blood pressure cuff includes:
(a) a flexible, hollow bladder adapted to surround the arm of the patient, which has an inside surface adapted to apply pressure to a surface of the arm above an artery, an outside surface opposite this inside surface, and an inlet line for receiving a fluid to cause the bladder to expand;
(b) at least one first sensor element arranged on a inside and/or outside surface of the bladder in a location adjacent the artery when the bladder is in its operative position surrounding the patient's arm; and possibly also
(c) at least one second sensor element arranged on a surface of the bladder that is opposite to the surface on which the first sensor element is disposed, and in a location adjacent the artery when the bladder is in its operative position surrounding the patient's arm. The sensor elements each comprise an elongate thin-film strip which transduces mechanical vibrations and produces an electrical signal representing these vibrations, normally in the range of 0 to 100 Hz.

### CROSS-REFERENCE TO RELATED APPLICATIONS

The present application discloses subjected matter which is related to that disclosed in the prior U.S. patent application Ser. No. 11/358,283, filed Feb. 21, 2006, and entitled “SYSTEM AND METHOD FOR NON-INVASIVE CARDIOVASCULAR ASSESSMENT FROM SUPRA-SYSTOLIC SIGNALS OBTAINED WITH A WIDEBAND EXTERNAL PULSE TRANSDUCER IN A BLOOD PRESSURE CUFF” (which prior application is incorporated herein by reference). The present application also claims priority from the U.S. Provisional Patent Application Ser. No. 60/777,418 (Sharrock 207), filed Feb. 28, 2006, and entitled “IMPROVEMENTS IN BLOOD PRESSURE CUFF SENSORS”.

### BACKGROUND OF THE INVENTION

The present invention relates to a blood pressure cuff of the type conventionally used in a sphygmomanometer to measure blood pressure according to the Korotkoff method. More specifically, the invention concerns blood pressure cuff that is specifically designed for non-invasive cardiovascular assessment of a patient based on the evaluation of signals obtained by a low frequency, wideband electrical transducer or sensor built into the cuff.

The signals recorded with a sensor placed on or in a blood pressure cuff are termed “supra-systolic” signals if the cuff pressure is above the subject's systolic blood pressure. Such signals can also be recorded when the cuff pressure is below systolic pressure. In all cases, the signals result from blood pressure energy transmissions and are dependent upon the subject's physiology.

When the heart pumps, a pressure gradient is generated within the cardiovascular system. This results in pulse pressure waves traveling peripherally from the heart through the arteries. Like any wave, they reflect back off a surface or other change in impedance. Arterial pulse waves reflect back from both the peripheral circulation and from the distal aorta when it becomes less compliant (Murgo, J. P. et al. “Aortic Input Impendence in Normal Man: Relationship to Pressure Waveforms”, Circulation 62, No. 1 (1980); Latham R. D. et al. “Regional Wave Travel and Reflections Along the Human Aorta”, Circulation 72 No. 6. (1985). These reflection waves are identifiable in arterial pressure tracings, but the exact timing and magnitude of the waves are difficult to discern. Nevertheless, they have been the basis of several commercial systems to assess reflectance waves. These systems measure arterial contours using applanation tonometry from the radial artery.

If a low frequency sensor is placed over the brachial artery beneath a blood pressure cuff and the cuff is inflated above systole, supra-systolic signals can be recorded (Blank, S. G. et al. “Wideband External Pulse Recording during Cuff Deflation” Circulation 77, No. 6 (1988); Hirai, T. et al. “Stiffness of Systemic Arteries in Patients with Myocardial Infarction” Circulation 1989; 80: 78-86; Denby, L. et al. “Analysis of the Wideband External Pulse” Statistics in Medicine, John Wiley & Sons (1994). These signals contain frequency components of less than 20 Hertz, which are non-audible. Supra-systolic low frequency signals provide clear definition of three distinct waves: an incident wave corresponding to the pulse wave and two subsequent waves. In 1996 Blank proposed that the second wave emanated from the periphery and the relative amplitude of this wave to the incident wave (K1R) was a measure of peripheral vascular resistance (PVR). He proposed a constant such that PVR could be measured from the ratio of the incident to the first reflectance wave. See U.S. Pat. No. 5,913,826, which is incorporated herein by reference in its entirety.

The second supra-systolic wave is, in fact, a reflectance wave from the distal abdominal aorta—most likely originating from the bifurcation of the aorta and not from the peripheral circulation as proposed by Blank. This has been verified in human experiments (Murgo, et al. and Latham et al., cited above) and in studies using pulse wave velocity (PWV) measurements. The relative amplitude of the first reflectance wave is now believed to be a measure of the stiffness, compliance, or elasticity of the abdominal aorta rather than peripheral resistance.

In the clinical experiments upon which Blank relied to formulate his hypothesis, changes in compliance were induced with epinephrine and epidural anesthesia. The changes in compliance were accompanied by changes in peripheral resistance. Thus, he saw a relationship between his K1R and PVR, but it was a co-variable and not a true association.

The third wave occurs at the beginning of diastole and is believed to be a reflection from the peripheral circulation or a secondary reflection from the iliac, after reflection from the subclavian branch. As such, it is a measure of peripheral vasoconstriction with superimposed secondary reflections. Supra-systolic signals can be utilized to measure compliance by relating the amplitude of the first wave (incident or SS1 wave) to the amplitude of the second wave (aortic reflection or SS2 wave). The degree of vasoconstriction can be assessed by measuring the amplitude of the diastolic or third wave (SS3 wave) and relating it to the SS1 wave. Amplitudes, areas under the curves, or other values calculated from the waves can be utilized as described in the aforementioned U.S. patent application Ser. No. 11/358,283. Such data have been analyzed by measuring amplitudes, ratios of amplitudes and time delays between waves.

The procedure for obtaining the signals for this method of non-invasive cardiovascular assessment, using the low-frequency, wideband external pulse transducer (sensor) was substantially as follows:
The sensor was placed against the patient's skin, over or near the brachial artery; and then
The blood pressure cuff was placed over the sensor and covered the sensor. In some cases adhesive tape or the like was employed to hold the sensor in place.

While this procedure yielded usable signals, the procedure was cumbersome in practice and the signals frequently contained unwanted noise due to artifacts and the like.

Certain known sensors for blood pressure cuffs, for example, the CardioDyne NBP2000 and the SunTech Medical Tango+, are utilized external to the cuff bladder and are held in place using removable adhesive or a specially designed pocket in the cuff. However, these sensors do not provide the low-frequency signals required for the cardiovascular assessment described above.

### SUMMARY OF THE INVENTION

It is therefore an object of the present invention to provide a blood pressure cuff, incorporating one or more low-frequency, wideband external pulse sensors, which is convenient to manufacture, apply and use in connection with a system and method for non-invasive cardiovascular assessment of a patient.

It is a further object of the present invention to provide a blood pressure cuff of the above-noted type, which is robust and produces clear, noise-free signals in a clinical environment.

These objects, as well as further objects which will become apparent from the discussion that follows, are achieved, in accordance with the present invention, by providing a blood pressure cuff comprising the following components:
A flexible, hollow bladder adapted to surround the arm of the patient, which has an inside surface adapted to apply pressure to a surface of the arm above an artery, an outside, free surface opposite this inside surface, and an inlet line for receiving a fluid to cause the bladder to expand; and
At least one “first” sensor element arranged on the inside and/or outside surface of the bladder in a location adjacent the artery when the bladder is in its operative position surrounding the patient's arm.
This first sensor element includes an elongate thin-film strip which transduces mechanical vibrations and produces an electrical signal representing these vibrations, normally in the range of 0 to 100 Hz.

In a preferred embodiment of the present invention, the blood pressure cuff also includes at least one “second” sensor element arranged on the inside and/or outside surface of the bladder that is opposite to the surface on which the first sensor element is disposed. This second sensor is preferably also in a location adjacent the artery when the bladder is in its operative position surrounding the patient's arm. This second sensor element also includes a thin-film strip which transduces mechanical vibrations and produces an electrical signal representing these vibrations.

According to a further preferred embodiment of the present invention, the sensor element(s) is (are) selected and configured to sense and transduce strain in the inside and/or outside surface of the bladder and to produce an electric signal representing this strain.

The preferred embodiments of the first and/or second sensor elements include one or more thin-film PVDF strips which utilize the piezoelectric effect to produce the electric signal.

There are various different configurations used to implement the present invention in practice (including types, shapes, positions, numbers of sensors and connecting electric circuits) of sensor elements on both the inside and the outside bladder surfaces. These are set forth in the following detailed description of the preferred embodiments of the invention as illustrated in the accompanying drawings.

### BRIEF DESCRIPTION OF THE DRAWINGS

FIG. 1 is a perspective view of a blood pressure cuff having both an inside sensor element and an outside sensor element according to a first preferred embodiment of the present invention.

FIG. 2 is a perspective view of a blood pressure cuff having multiple inside sensor elements according to a second preferred embodiment of the present invention.

FIG. 3 is a schematic view of a two-channel sensor system for a blood pressure cuff according to a third preferred embodiment of the present invention.

FIG. 4 is a block diagram of an electronic circuit for a blood pressure cuff according to the invention, which circuit converts the analog outputs of the sensor elements into digital signals and transmits the digital data to a computer for storage, retrieval and analysis.

FIG. 5 is a representational diagram of a connector and plug for both a pneumatic line and electronic leads in a blood pressure cuff according to the invention.

FIG. 6 illustrates typical sensor signals during blood pressure cuff deflation (volts vs. time in seconds) for both an inside sensor element and an outside sensor element.

FIG. 7 is a graph of a high frequency inside sensor signal (right axis) and cuff pressure (left axis) vs. time in seconds.

FIG. 8 is a graph showing positional and interbeat variablility of an outside sensor element.

FIG. 9 is a graph of sensor element signals showing a succession of mean supra-systolic beats.

FIG. 10 is a graph of sensor element signals showing a mean sub-diastolic beat.

FIG. 11 is a representational diagram of the components of a pressure wave represented by the sensor element output.

FIG. 12 is a representational diagram showing an initial step in the manufacture of a blood pressure cuff according to the invention.

FIG. 13 is a representational diagram showing a next following step in the manufacture of a blood pressure cuff according to the invention.

FIG. 14 is a representational diagram showing still another next step in the manufacture of a blood pressure cuff according to the invention.

FIG. 15 is a representational diagram showing a final step in the manufacture of a blood pressure cuff according to the invention.

### DESCRIPTION OF THE PREFERRED EMBODIMENTS

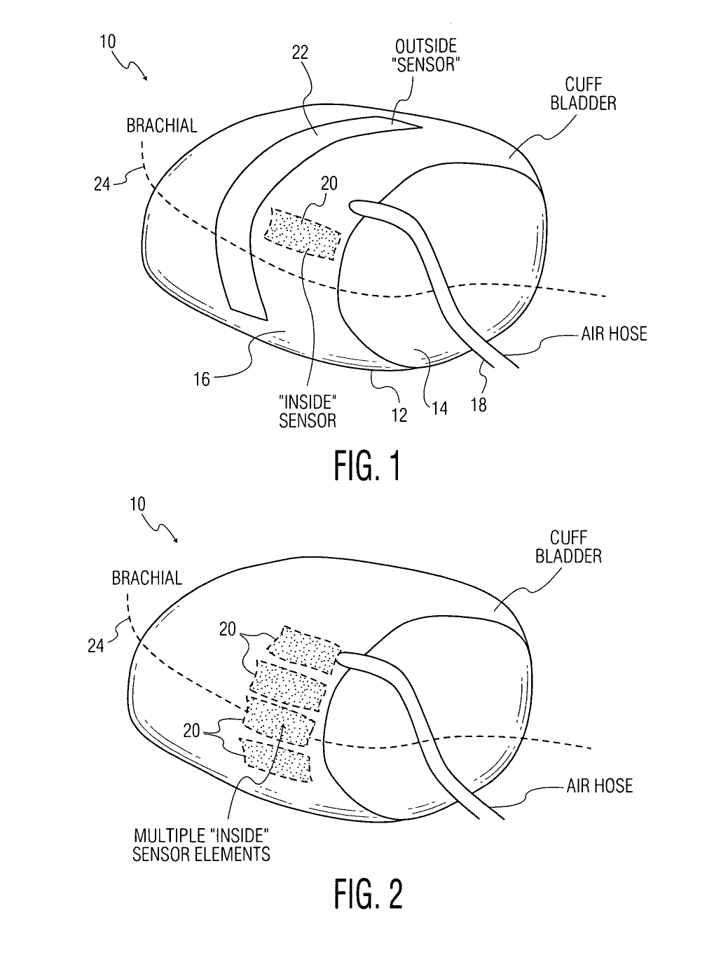

The preferred embodiments of the present invention will now be described with reference to FIGS. 1-15 of the drawings. Identical elements in the various figures are designated with the same reference numerals.

### General Principles:

The general layout of the blood pressure cuff according to the present invention is illustrated in FIG. 1. The cuff 10 includes a flexible, hollow bladder 12 adapted to surround an arm of a patient, which has an inside external surface 14 adapted to apply pressure to the surface of the arm, an outside external surface 16 opposite the inside surface, and an inlet tube 18 for receiving a fluid to cause the bladder to expand.

An “inside” sensor element 20 (shown in a dotted outline) is affixed, e.g., by a suitable adhesive or by welding, to the inside surface 14 of the cuff bladder that is pressed against the arm when the cuff is inflated. An “outside” sensor element 22 is affixed to the outside surface 16 of the cuff bladder; e.g., by an adhesive. Alternatively, the sensors may be held in place by applying a cover film, which, in turn, is affixed to the bladder.

These sensors may be of different shapes, sizes and materials and there may even be multiple inside and/or outside sensor elements. The sensors are primarily designed to instrument the cuff itself, not the arm, nor specifically the brachial artery 24. The sensors are preferably made of PVDF film and are designed to transduce strain (i.e. elongation) in the cuff wall and produce an electric signal representing this strain. The shape and position of the sensors can be tailored to capture specific modes of cuff wall movement. This in turn reflects a specific aspect of the arterial pulse. For example:
A long, narrow outside sensor wrapped circumferentially (as shown in FIG. 1) is most sensitive to very small signals, such as those generated at supra-systolic and sub-diastolic cuff pressures. The geometry of the cuff acts as an amplifier and produces electrical signals with a high signal to noise ratio.
Inside sensors placed axially towards the distal edge of the cuff (again shown in FIG. 1) appear most sensitive to the Korotkoff sounds produced at systolic and diastolic pressures.

The shape and position of sensors can be tailored to reduce the positional dependence of cuff or sensor placement. For example:
A sensor that wraps circumferentially around a large portion of the cuff bladder is less dependent on rotational cuff position relative to the brachial artery.
Multiple, possibly connected, axially orientated sensor elements can be used inside the cuff to achieve a similar effect, as shown in FIG. 2.

The shape and position of sensors can be tailored to help reduce sensitivity to, or identify, signal artifact. It is envisaged that sensors could be placed to pick up modes of cuff movement that are primarily excited by arm movement, for example. These could be used to identify periods of excessive movement when processing signal from the other sensors.

### Alternative Configurations:

The air-filled blood pressure cuff and sensor arrangements described above are only a special (though perhaps practically convenient) case for an apparatus to measure pressure fluctuations in a vessel (in this case the brachial artery 24) embedded in a soft, incompressible medium. Related configurations may allow more accurate determination of the internal fluid properties (pressure, flow, etc.). For example:
A liquid, gel or something other than gas-filled cuff may transmit higher-fidelity pressure fluctuations.
Use of a hard outer shell rather than the outside wall of the cuff bladder could allow more consistent, more meaningful signal interpretation.
Actively controlling the “cuff” pressure would allow the input of known, calibrated signal into artery. The response would then allow much more analysis and information than is currently the case. It might even be possible to construct a sort of “arterial system Doppler.”
Although PVDF thin-film transducers are convenient to measure strain in the cuff wall, other sensor technologies (e.g. bridge-based strain gauges) may also be used.

### Specific Implementation:

In a preferred embodiment and presently the best mode of practicing the invention, the instrumented cuff includes two inside sensor elements and one outside sensor element, whose outputs are telemetered to the computer via two separate, independent channels. This implementation has been designed to have the following features:
Low positional sensitivity.
Low channel count—two channels keeps the electronics and signal processing simple.
High sensitivity to supra-systolic signals.
High sensitivity to “Korotkoff” vibrations generated at systolic and diastolic pressures.
Inexpensive and high manufacturability.

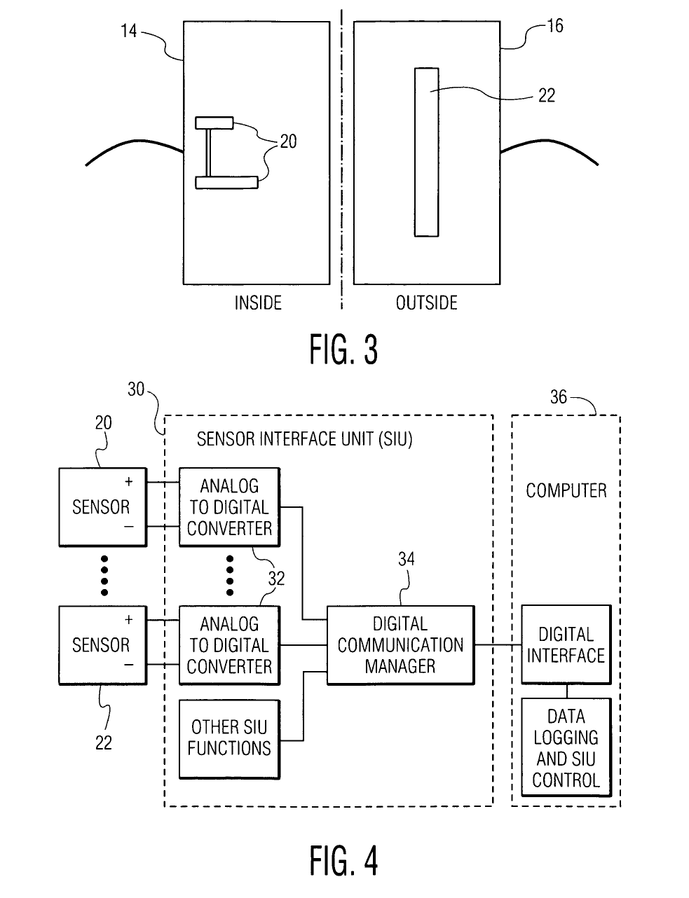

FIG. 3 shows the two sides of a cuff bladder for this embodiment. The blood pressure cuff may, for example, be of the type available from manufacturers such as Trimline Medical Products Corp., which sells cuffs under the brand names Bainbridge and Kuff-Link.

The outside sensor element is a single long element fixed in the circumferential direction available commercially from Measurement Specialties Inc. (“MSI”). The MSI DT4-52K/L piezo film elements are of a suitable size, being 19×171 mm, and 70 μm thick.

The inside sensor element is composed of two connected elements parallel to the axial direction of the artery and spaced approximately 40 mm apart to their center lines. They are positioned about 15 mm from the distal edge of the cuff bladder. The two elements are of different lengths. In experiments, MSI DT1-52K/L and DT2-52K/L sensing elements were used, being 12 mm wide and 30 and 62 mm long, respectively, and 70 μm thick. The negative terminals of the two elements may be connected together, and the sensed voltage is that generated from the positive terminal of one element to the positive terminal of the other. Multiple sensor elements (both inside and outside) may be connected together in any number of ways, including series connections and parallel connections.

The theory of operation of the configuration shown is as follows:

The outside sensor is designed to be most sensitive to small signals radiating from the artery, such as supra-systolic signals.
Such signals are amplified by the cavity in the bladder and result in stress and strain within the cuff wall.
The outside wall of the bladder is most free to move (unconstrained by the arm) and the sensor measures this strain.
The voltage generated in the piezo element is thus proportional to strain, which in turn is related to the pressure fluctuation, volume of air in the cuff and elasticity of the cuff wall.
The length of the outside sensor reduces the sensitivity to cuff position and local deformations in the cuff wall.
The high aspect ratio (relatively narrow width) determines that the sensor element is most sensitive by far to strain in the circumferential direction.

The inside sensor element is designed to detect Korotkoff sounds for determination of systolic and diastolic pressure.
In a similar manner to a stethoscope, the sensors are on the inside of an air-filled cavity (cuff bladder) and pressed against the brachial artery.
The sensors are orientated parallel to the artery to maximize the mechanical coupling (and hence sensitivity) between the sensor and the artery.
The sensors are placed at the distal edge of the cuff to detect turbulence flowing under the cuff.
Although one sensing element would be sufficient, two are used to reduce positional sensitivity. As long as the cuff is placed with the air hose near the brachial artery, one or other of the sensor elements will be sufficiently “close by.”
The two sensor elements are connected with reverse polarity. That is, the overall signal is the difference between the voltages generated by the two sensors. Given the spacing of the sensors, this filters out “common-mode” voltages and amplifies any phase differences between the two elements, which will presumably have a greater effect on higher frequency signals, such as those of interest in detecting Korotkoff sounds.
The two sensor elements are made different lengths to give them different frequency responses and further accentuate phase differences. According to the theory of piezoelectric-film materials, the voltage generated is unaffected.

### Electric Circuits and Connecting Plug:

FIG. 4 is a block diagram of the interface between the sensors and a computer, which is called the “sensor interface unit” (SIU) 30. Multiple sensors may be serviced by corresponding analog-to-digital converters (ADC) 32. These ADCs provide a digital representation of the sensors' analog signal to a digital communication manager (DCM) 34. The DCM marshals the ADC output (for example, in SPI format) to a digital transmission protocol understandable by the computer 36 (which may be, for example, RS232 or USB). The DCM may also process digital data sent from the computer 36 to other functions of the sensor interface unit 38, for example, to select ADC units for each conversion. In this way, the analog signal path length is minimized.

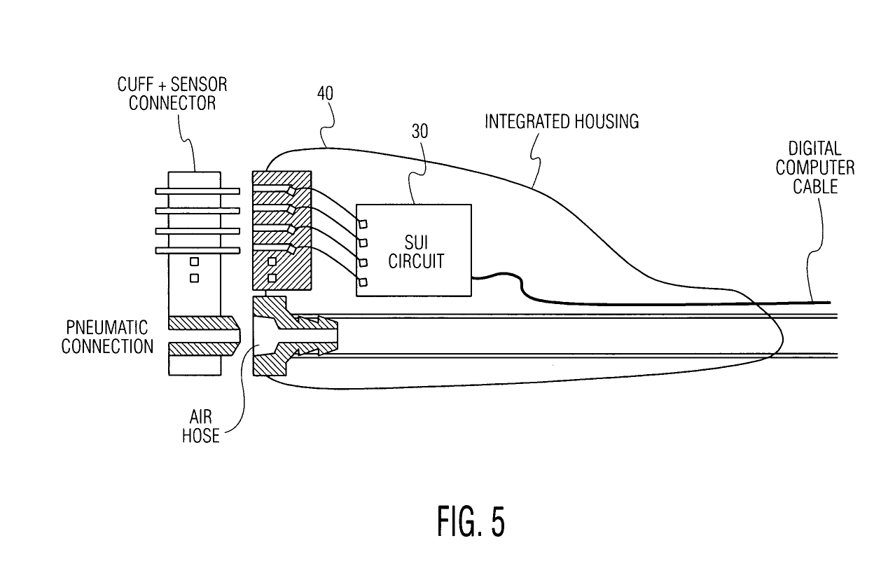

FIG. 5 shows a plug 40 in a common housing assembly 40 integrating both electrical and fluid connections for the blood pressure cuff. This plug is connectable to a connector assembly 42 attached to the bladder. The actual physical layout may vary somewhat from that shown which is intended to illustrate the general principle of operation. With this plug and connector, both electrical and fluid connections are made at the same time when the cuff and sensor are connected. The SUI can be powered from the digital computer cable or by a separate parallel wire.

### Output Signals:

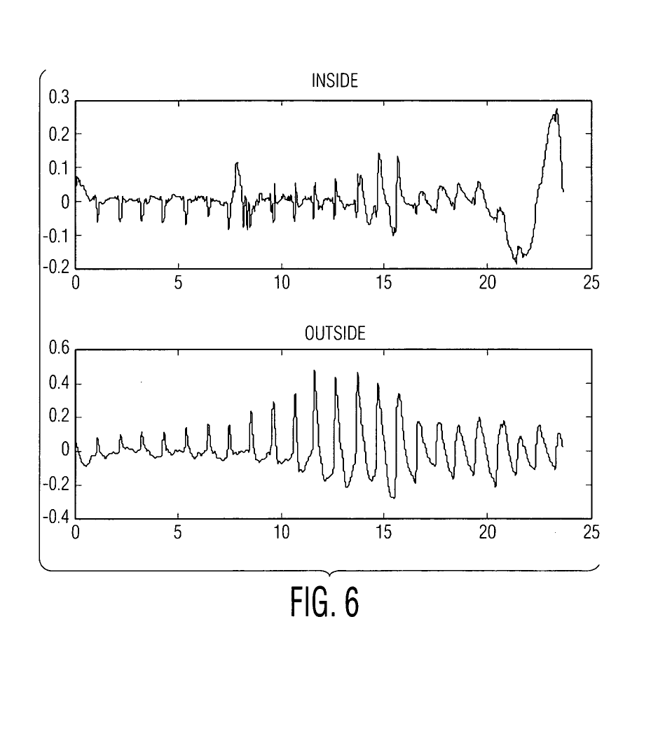

Typical signals from the instrumented cuff described above, received from an inside sensor element 20 and an outside sensor element 22, are shown in FIG. 6 for a cuff deflation cycle. The signals shown have been band-pass filtered in both directions (i.e. zero phase change) using a second order Butterworth digital filter with corner frequencies at 0.25 and 30 Hz.

### Waveform Interpretation:

There are two major factors contributing to improvements in the interpretation of the waveform. First, the sensor technology allows a much more information-rich signal. This is mainly due to a vastly improved low-frequency response. Second, advances in the understanding of the physiology, particularly due to engineering modeling efforts, aid in the interpretation of the waveforms.

### Determination of Systolic and Diastolic Signals:

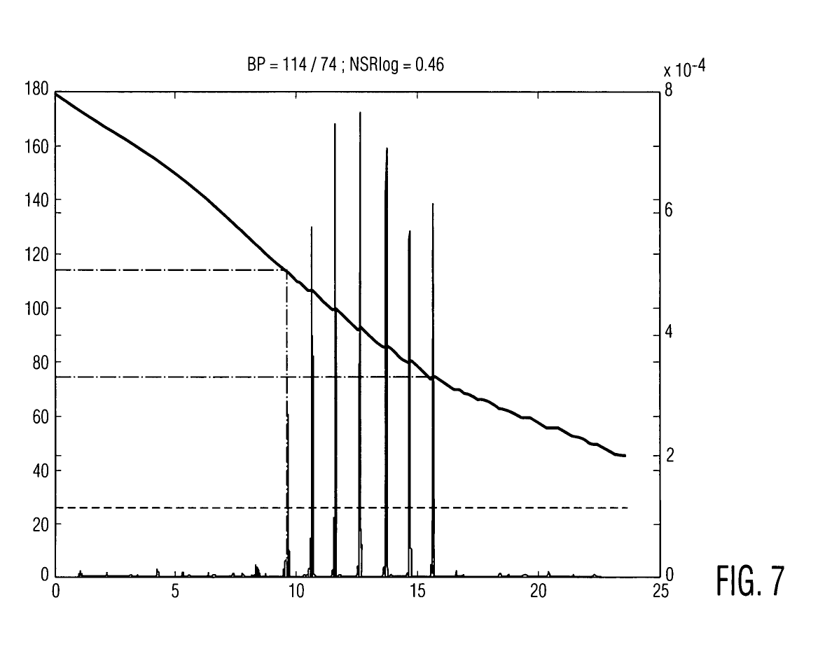

Calculating the power of the high-pass-filtered inside sensor signal produces a graph as shown in FIG. 7. The cuff pressure is also shown and a feasible blood pressure has been calculated using a threshold method. The filter corner frequency was 8.5% of the Nyquist, i.e. enough to remove the DC component.

Although not immune to noise, FIG. 7 is a typical tracing of signals obtained through actual experiments. Furthermore, the result is broadly reproducible for a wide range of sensor positions within at least a 90° arc.

### Positional Independence of Supra-Systolic Signals:

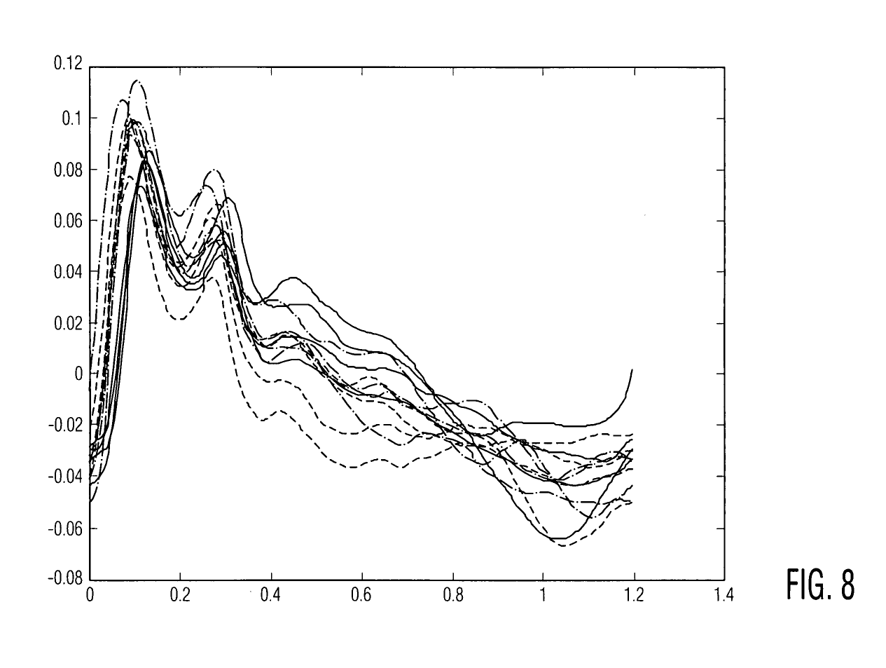

In a similar manner, the outside sensor also exhibits robustness with respect to cuff placement. FIG. 8 shows supra-systolic signals recorded between 140 mmHg and 150 mmHg from a subject over a 5 minute period. The plot shows four beats from each of three recordings. The three recordings were with the cuff at approximately 0° and ±70° from “ideal” placement. It can be seen that respiratory variability is on par with positional variability using this sensor.

### Waveform Morphology:

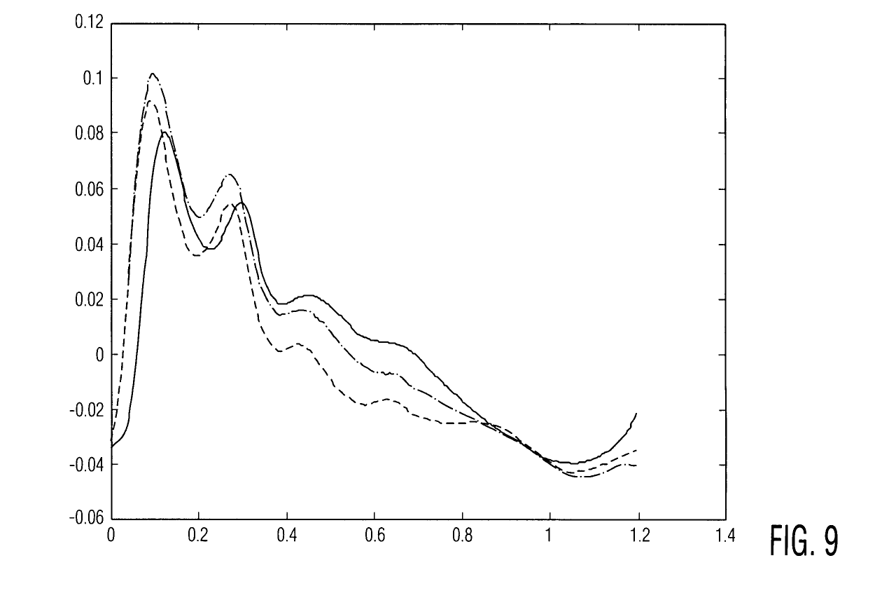

The mean beats corresponding to those shown in FIG. 8 are shown in FIG. 9. The shape of the waveform from the sensor elements of the present invention is very similar to an invasive radial pressure waveform. Furthermore, at sub-diastolic pressures, the waveform morphology for the outside sensor is consistent.

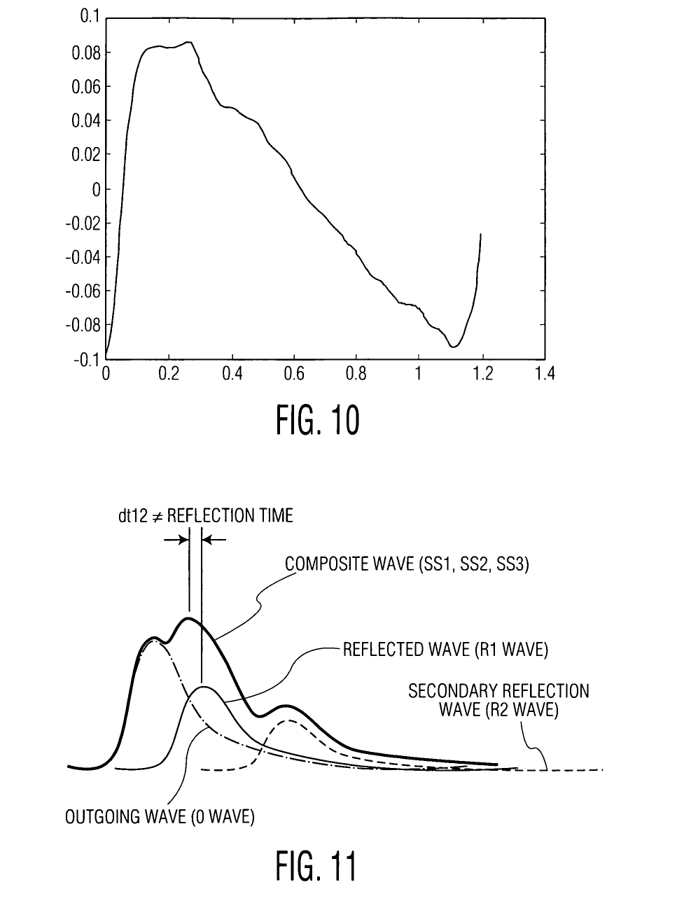

FIG. 10 shows a mean sub-diastolic pulse wave, taken at 50 mmHg. It is evocative of a central arterial pressure waveform.

If the correlation with invasive pressure can be proven then the new sensor provides a number of opportunities:

The waveform voltage axis can be scaled between systolic and diastolic pressure, and displayed as a pressure.

Models and analysis of invasive pressure waveforms can be applied to the externally collected signal.

The difference between supra-systolic and sub-diastolic signals may contain important information. For example, the difference may be indicative of blood flow through the brachial artery. It may also allow continuous BP monitoring using the sub-diastolic waveform and some transformation, which could be calibrated using a traditional NIBP cycle.

### Model-Based Interpretation:

In conjunction with the richer signal, efforts directed to the physical (engineering) modeling of the arterial system can be used to better interpret the waveform for clinical use.

Specific findings of the modeling work are as follows:
Physical properties indicate that dynamics are mostly linear. Additionally, damping is relatively insignificant and dynamic impedance is relatively flat across low frequency range of interest. This means that reflected waves have very similar shape to the incident waves. It also means that the SS1 wave is basically unchanged in shape by the propagation from the heart.
Time delays and reflection coefficients are similar to those observed in practice.
The SS3 waveform will be a combination of secondary reflections, and possibly reflections from more peripheral sites. In any case, the more peripheral reflections will be of less significance than originally believed.

These findings imply the following, with reference to FIG. 11, which illustrates components of the composite SS1, SS2 and SS3 waveform:

The first part of the SS1 wave is equivalent to the first part of the outgoing O wave and thus a good indicator of cardiac performance.

The R2 wave is a scaled version of the R1 wave, which, in turn, is a similarly scaled version of the O wave. This knowledge aids in determining the component waves from the composite wave.

The delay between the O and R1 waves is a better indicator of pulse wave velocity than the time between the SS1 and SS2 peaks (i.e. dt12).
The augmentation index (AI) is a non-linear function of both R1 to O wave height ratio, reflection time delay, and wave shape. The current, common understanding of AI, “early” and “late” systolic pressures, etc., is insufficient.
The revised model of the arterial system has implications on the interpretation of the timing and meaning of the dichrotic notch.

### Cuff Manufacture:

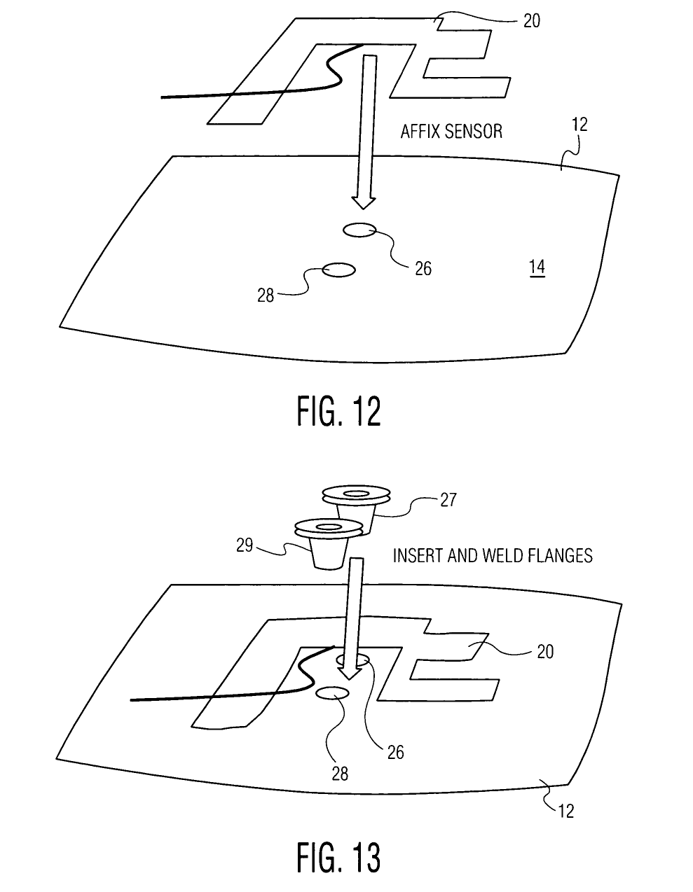

FIGS. 12-15 are a sequence of diagrams illustrating a possible manufacturing process for integrating a sensor element into a cuff. This process closely follows the standard cuff manufacturing technique for non-instrumented cuffs. A standard two-hole cuff can be used, except that one flange is used to provide a gasket around the sensor wire(s).

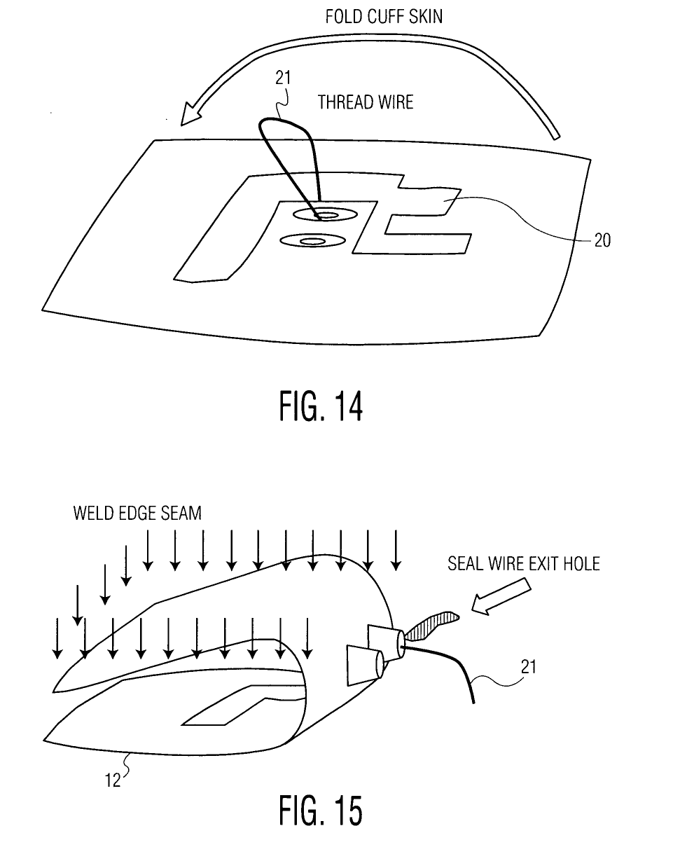

In FIG. 12 the sensor element is affixed to a surface 14 of the cuff material 12, e.g., by means of an adhesive or by welding, or both. Alternatively, the sensor element may be held in place by a cover strip of material which, in turn, is attached to the cuff material 12. The sensor 20 is shown as having an arbitrary shape. FIG. 13 illustrates the insertion of the flanges 27 and 29 into the two holes 26 and 28, respectively, in the material 12, where they are welded in place. In FIG. 14 the signal transmission wire 21 from the sensor 20 is threaded through one of the flanges 27 and the cuff material 12 is folded over to form the bladder. Finally, as shown in FIG. 15, the edges of the material 12 are welded or gluded together and the wire is sealed in the exit hole.

Sealing the signal transmission wire may be achieved by a number of methods, including:
Hot-melt sealant applied to the hole;
Heat-shrink tubing applied around the flange protrusion;
A custom designed gasket;
Wireless or optical signal transmission through a window in the cuff; and
Using a transmission wire or flexible circuit capable of being heat-welded during the fabrication process.

### Summary:

In summary, the present invention concerns a blood pressure cuff which integrally incorporates one or more sensor elements in the cuff bladder. This arrangement has a number of important advantages:
It allows the cuff and sensor to be a single unit;
The sensor can be incorporated at time of manufacture, increasing the reliability of placement and decreasing complexity for the user.
No protrusions from the cuff create increased comfort for the wearer;
More of the cuff surface is available for a hook-and-loop fastener, markings, branding etc.;
The cuff wall is inherently gas and liquid impermeable, protecting any sensitive and/or electronic components integrated into the sensing elements; and
If the cuff material incorporates a conductive layer (for example, internally) then it can act as a shield against electromagnetic interference.

Finally, the resulting blood pressure cuff has a number of advantageous features that are especially useful in measuring supra-systolic signals in addition to the conventional Korotkoff sounds. These are:
Instrumented cuff: The sensors are used to specifically measure modes of energy transmission through the cuff, rather than through the air chamber (oscillometric) or the arm itself (auscultatory).
Integrated sensors: The cuff can be manufactured with sensors as an integral part forming a “smart electronic cuff”. This is especially relevant to disposable cuffs, where inexpensive sensors can be used.
Flexible circuits within the cuff: The film sensors are manufactured on a flexible substrate. This substrate may be bonded or form part of a flexible printed circuit board, carrying electronic components within the cuff. Such components can provide active functionality such as signal conditioning, digitization, electronic cuff serial number/model identification (e.g. Adult cuff, used 12 times to date, expires in 3 months, etc.), sensor integrity check/calibration, etc.
Air connector with electrical contacts: Currently, cuff connectors are air-only and if electrical connections are required, they are made separately. One connector integrating both electrical and air would be a significant usability enhancement.
Digitized signal transmission: Transmitting analog signals over long wires promotes interference and noise. Digitizing the analog signal at the cuff-end (either in the cuff or in the connector) allows transmission via a digital protocol, such as USB or even wireless. Digitizing the signal from multiple sensors also means a reduced wire count and therefore a more flexible line between the cuff and the device.
Elegant: The design is readily manufactured, inexpensive and inconspicuous. Usability is as good as oscillometric techniques.
Functional: The design allows significant improvements in signal quality, noise rejection and patient comfort. There is also great flexibility in sensor design.
Informative: The signal, coupled with model-based interpretation, allows deep insight into physical, physiological parameters of the arterial and cardiac systems.

There has thus been shown and described novel improvements in blood pressure cuffs which fulfill all the objects and advantages sought therefor. Many changes, modifications, variations and other uses and applications of the subject invention will, however, become apparent to those skilled in the art after considering this specification and the accompanying drawings which disclose the preferred embodiments thereof. All such changes, modifications, variations and other uses and applications which do not depart from the spirit and scope of the invention are deemed to be covered by the invention, which is to be limited only by the claims which follow.

What is claimed is:

1. Blood pressure cuff apparatus for constricting an artery in a peripheral limb of a mammal and sensing cardiovascular performance of the mammal, said blood pressure cuff comprising, in combination:
(a) a flexible, hollow bladder adapted to surround the limb with the artery, said bladder having an inside external surface adapted to apply pressure to a surface of the limb, an outside external surface on the side opposite said inside surface, and an inlet for receiving a fluid to cause the bladder to expand;
(b) at least one first sensor element disposed on at least one of said inside surface and outside surface of the bladder in a location adjacent said artery when said bladder is in an operative position surrounding said limb, said first sensor element including an elongate thin-film strip for transducing mechanical vibrations and producing electrical signals representing said vibrations.

2. The blood pressure cuff recited in claim 1, wherein said limb of a mammal is an arm of a human patient and said artery is a brachial artery.

3. The blood pressure cuff defined in claim 1, wherein said first sensor element includes a piezo-electric, thin film material.

4. The blood pressure cuff defined in claim 3, wherein said material is PVDF.

5. The blood pressure cuff defined in claim 1, wherein said first sensor element transduces strain in said surface of the bladder on which it is disposed and produces an electric signal representing said strain.

6. The blood pressure cuff defined in claim 1, further comprising at least one second sensor element disposed on at least one of said inside surface and said outside surface of the bladder that is opposite to the surface on which said first sensor element is disposed, said second sensor element including an elongate thin-film strip which transduces mechanical vibrations and produces an electrical signal representing these vibrations.

7. The blood pressure cuff defined in claim 6, wherein said second sensor element includes a piezo-electric, thin film material.

8. The blood pressure cuff defined in claim 7, wherein said material is PVDF.

9. The blood pressure cuff defined in claim 6, wherein said second sensor element transduces strain in said surface of the bladder on which it is disposed and produces an electric signal representing said strain.

10. The blood pressure cuff defined in claim 1, wherein said elongate strip of said first sensor element is disposed substantially parallel to a direction of the artery when said blood pressure cuff is positioned on said limb.

11. The blood pressure cuff defined in claim 1, wherein said first sensor element comprises at least two elongate strips which extend substantially parallel to each other.

12. The blood pressure cuff defined in claim 11, wherein said at least two elongate strips extend substantially parallel to a direction of the artery when said blood pressure cuff is in an operative position surrounding said limb.

13. The blood pressure cuff defined in claim 11, wherein said at least two elongate strips extend substantially transverse to a direction of the artery when said blood pressure cuff is in an operative position surrounding said limb.

14. The blood pressure cuff defined in claim 11, wherein said at least two elongate strips are connected in series.

15. The blood pressure cuff defined in claim 11, wherein said at least two elongate strips are connected in parallel.

16. The blood pressure cuff defined in claim 1, wherein said bladder has a proximal end and a distal end when it surrounds said limb, and wherein said first sensor element is disposed adjacent the distal end of said bladder.

17. The blood pressure cuff defined in claim 6, wherein said elongate strip of said second sensor element is disposed substantially parallel to a direction of the artery when said blood pressure cuff is positioned in an operative position surrounding said limb.

18. The blood pressure cuff defined in claim 6, wherein said elongate strip of said second sensor element is disposed substantially transverse to a direction of the artery when said blood pressure cuff is positioned in an operative position surrounding said limb.

19. The blood pressure cuff defined in claim 1, wherein said first sensor element is attached to said surface of the bladder by means of at least one of an adhesive and welding.

20. The blood pressure cuff defined in claim 1, wherein said first sensor element is held in place by a cover film which is attached to the surface of the bladder.

21. The blood pressure cuff defined in claim 1, wherein each first sensor element is connected to an analog-to-digital (A/D) converter.

22. The blood pressure cuff defined in claim 21, wherein the A/D converter is connected to a digital communication manager (DCM) which converts the output of the A/D converter to a standard communication protocol for transmission to a digital computer.

23. The blood pressure cuff defined in claim 22, wherein the first sensor element is electrically connected to a connector jack, and wherein the A/D converter and DCM are disposed in a plug which is connectable electrically to the connector jack.

24. The blood pressure cuff defined in claim 23, wherein the plug is connectable by a cable to a computer.

25. The blood pressure cuff defined in claim 24, wherein the connector jack also has a receptacle for a fluid connection to the bladder, and wherein the plug is connectable fluidically to the receptacle.

26. The blood pressure cuff defined in claim 25, wherein the plug is connectable to a source of fluid under pressure by a flexible hose.
